(19) United States
(12) Patent Application Publication
(10) Pub. No.: US 2001/0046523 A1
(43) Pub. Date: Nov. 29, 2001

(54) Ginger-based herbal composition for promoting health and methods of using same

(76) Inventors: Thomas Newmark, St. Louis, MO (US); Paul Schulick, Brattleboro, VT (US)

Correspondence Address: Mary A Montebello, Patton Boggs LLP, 2550 M Street N W, Washington, DC, 20037, US

(21) Appl. No.: 09/513,224

(22) Filed: Feb. 25, 2000

(51) Int. Cl. A61K 35/78

(52) U.S. Cl. 424/756

(57) ABSTRACT

An herbal composition which can be used to promote stomach, liver and intestinal health; reduce inflammation; support blood platelet health and cardiovascular function; and provide antioxidant benefits; contains therapeutically effective amounts of a supercritical extract of ginger (preferably of certified organic ginger) and either a regular or supercritical (preferably supercritical) extract of rosemary. The herbal composition is preferably administered orally or parenterally.

### BACKGROUND OF THE INVENTION

This invention relates to a ginger-based herbal composition. More particularly, this invention relates to a ginger-based herbal composition which can function as a dietary supplement to promote a full spectrum of health benefits including, e.g., normal cell growth; stomach, liver, intestinal and blood platelet health; and reduction in inflammation of bones and joints. The present invention further relates to methods of promoting the aforementioned health benefits using a ginger-based herbal composition.

Ginger is widely recognized as one of the most well researched and efficacious herbs for multiple applications including support for stomach, liver and intestinal health, reducing inflammation, oxidative stress protection, cardiovascular health and anticancer effects.

Intensive research is now underway on a particular enzyme in the eicosanoid cascade called 5-lipoxygenase (5LO). Widespread acceptance is now being amassed which identifies key roles for this enzyme, particularly as it relates to prostate health. Authoritative research indicates that inhibition of this enzyme can cause massive and rapid apoptosis of prostate cancer cells. Unfortunately pharmaceutical approaches to curtailing this enzyme can lead to significant side effects.

Ginger is known to be able to inhibit the enzyme 5-lipoxygenase. See, e.g., U.S. Pat. No. 5,763,673. This enzyme promotes the synthesis of leucotrienes which are biological agents which exhibit strong physiological actions, such as leucocyte chemotaxis, acceleration of blood vessel permeability, and the like, and are thus known to have significant relations to inflammatory reactions. Research from databases combing international studies indicates that ginger contains the highest number of constituents which can inhibit 5LO. See, e.g., Chem Pharm Bull (Tokyo) 1992 February;40(2):387-91 Inhibition of prostaglandin and leukotriene biosynthesis by gingerols and diarylheptanoids. Kiuchi F, Iwakami S, Shibuya M, Hanaoka F, Sankawa U Faculty of Pharmaceutical Sciences, University of Tokyo, Japan; and Nippon Yakurigaku Zasshi 1986 October;88(4):263-9 [Pharmacological studies on ginger. IV. Effect of (6)-shogaol on the arachidonic cascade]. Suekawa M, Yuasa K, Isono M, Sone H, Ikeya Y, Sakakibara I, Aburada M, Hosoya E.

Currently, the commercial marketplace is unaware of the potential for ginger to positively affect prostate health and reduce cancer progression through this mechanism.

Although ginger is known to inhibit 5-lipoxygenase, it is continually desirable to provide a ginger-based herbal formulation which can exhibit a stronger 5-lipoxygenase inhibiting activity and, thus, a stronger anti-inflammatory effect. It is further desirable to provide a ginger-based herbal composition which not only exhibits a strong anti-inflammatory effect but also provides other important therapeutic benefits such as, e.g., the promoting of stomach, liver and intestinal health; antioxidant activity; and support for blood platelet health and good cardiovascular function.

Accordingly, a primary object of this invention is to provide a ginger-based herbal composition which has improved anti-inflammatory properties.

A further object of this invention is to provide a ginger-based herbal composition which, in addition to having improved anti-inflammatory properties, can also promote stomach, liver and intestinal health, support blood platelet health and good cardiovascular function, and provide antioxidant activity.

Another object of this invention is to provide a ginger-based herbal composition having the characteristics set forth in the preceding objects, wherein the composition can be orally or parenterally administered.

A further object of this invention is to provide methods of providing the health benefits recited in the preceding objects using the herbal composition of this invention.

These objects and others are achieved in the present invention.

### SUMMARY OF THE INVENTION

The present invention provides a unique herbal formulation which is designed to highlight ginger's 5-LO inhibiting and antiulcer constituents and preserve them by utilizing a specially extracted formulation of rosemary. Ginger constituents preserved by rosemary are the gingerols (20% concentration). Through unique antioxidant mechanisms, carnosic acid in rosemary prevents the conversion of the gingerols to less active or researched shogaols.

A further novel feature of the herbal formulation of this invention is that it incorporates with the pungent gingerols the significant antiulcer agent zingiberene in guaranteed potency of 5% levels.

In a novel feature of a preferred embodiment of the present invention, the ginger supercritical extract is the supercritical extract of certified organic ginger, which provides higher levels of essential oil and pungent compounds.

Another novel feature of the present invention is that supercritical-solvent free extraction technology is used in association with 5-lipoxygenase inhibition. This technology allows for highest potency of active compounds, as much as 250 times the potency of original fresh plant material

The herbal formulation of the present invention contains therapeutically effective amounts of a supercritical extract of ginger (preferably a supercritical extract of certified organic ginger) and a regular or supercritical extract (preferably supercritical extract) of rosemary.

The herbal composition of this invention provides a full spectrum of therapeutic benefits. For example, the composition functions as an effective prostaglandin modulator, thereby naturally reducing inflammation throughout the body. Furthermore, the composition promotes fat-digesting bile and starch-digesting saliva, as well as the growth of beneficial intestinal microorganisms, which are vital for intestinal health. The composition of this invention also contains numerous anti-aging and cardiotonic constituents that inactivate oxygen free radicals, some of these constituents being up to forty times more effective than Vitamin E.

The herbal composition of this invention is preferably administered orally or parenterally. Thus, the present invention further provides a composition composed of the herbal active-ingredient composition in combination with a pharmaceutically acceptable oral or parenteral carrier. Preferably, the composition is administered orally, more preferably as a soft gel capsule.

The present invention also provides methods of using the herbal formulation to provide the therapeutic benefits recited above.

### DETAILED DESCRIPTION OF THE INVENTION

As stated above, the present invention provides a ginger-based herbal composition capable of promoting a full spectrum of health benefits. In preferred embodiments of the invention, the ginger used is a certified organic ginger.

The active-ingredient herbal composition of the present invention contains a supercritical extract of ginger (preferably of certified organic ginger) and either a regular or supercritical extract of rosemary. Preferably, supercritical extracts of the rosemary are used in this invention.

As used herein, the term “extract” is intended to mean a concentrate of water-soluble and/or alcohol-soluble plant components from the portion of the plant extracted and can be in aqueous or powdered form. The term “supercritical extract” as used herein means an extract obtained using a supercritical extraction process as discussed later herein. An extract which is not obtained using a supercritical extraction process is referred to herein as a “regular extract” so as to distinguish these extracts from supercritical extracts.

The supercritical extract of ginger used in the present invention is preferably taken from the rhizome, while the rosemary supercritical or regular extract is preferably obtained from the leaves and essential oil of the plant.

Supercritical extraction of the ginger and rosemary plant portions can be carried out according to known supercritical extraction methods. Such methods are disclosed, e.g., in U.S. Pat. Nos. 5,932,101 and 5,120,558, which are hereby incorporated by reference herein.

U.S. Pat. No. 5,932,101 discloses a supercritical extraction process wherein an extraction solvent and a fluid feed are supplied with a countercurrent flow to an extraction column. The extraction solvent contains a dense gas (e.g., carbon dioxide), and the fluid feed contains at least one solute (e.g., an herb) and a carrier fluid (e.g., water). The solute is selective to the extraction solvent with respect to the carrier fluid. The carrier fluid contains at least one component which is barely soluble in the extraction solvent and substantially immiscible with the extraction solvent so as to provide two phases. The fluid feed and the extraction solvent intimately contact one another in the column for a sufficient amount of time to extract the solute from the carrier fluid to the extraction solvent. The column operates in an enhanced solubility region having a pressure of between 450 and 1200 bar and a temperature of between 50° C. and 300° C. The column has a diameter greater than about 3.5 centimeters and a height to diameter ratio of greater than about 5. A raffinate containing the carrier fluid is removed from the column, as is an extract containing the extraction solvent and the solute. The combination of pressure and temperature is sufficient for the solubility of the solute in the extraction solvent to be at least 250% by weight greater than the solubility of the solute in the extraction solvent at the same operating temperature but at 200 bar pressure. Additionally, the solute may be separated from the extraction solvent in a phase separation device such as a decanter, a coalescer, a cyclone and a second extraction column.

The supercritical extraction process disclosed in U.S. Pat. No. 5,120,558 involves grinding a spice or herb and then extracting the ground spice or herb with supercritical fluid carbon dioxide under a pressure of from about 400 bar to about 600 bar and at a temperature of from about 80° C. to about 120° C. At least one oleoresin fraction is precipitated from the loaded supercritical fluid under a pressure of from about 280 bar to about 380 bar and at a temperature of from about 80° C. to about 100° C. Additional oleoresins may be obtained by next adjusting the pressure of the supercritical fluid to from about 100 bar to about 200 bar within the same temperature range of 80° C. to 100° C., and finally by reducing the pressure to from about 30 bar to about 50 bar and the temperature to from about 0° C. to about 30° C.

A regular extract of rosemary can be obtained using either conventional or supercritical extraction techniques. Suitable conventional extraction techniques are disclosed, e.g., in U.S. Pat. Nos. 5,891,440; 5,874,084; and 5,908,628; all of which are hereby incorporated by reference herein.

For example, the regular extract can be prepared by contacting the herb with an appropriate solvent to form the extract. To make the extract suitable for oral administration, the solvent used must be substantially non-toxic to the subject so that there is no untoward level of adverse side effects, such as toxicity, irritation, allergy or hypersensitivity responses. The level of any such side effects should be commensurate with acceptable risk/benefit ratios. Examples of such substantially non-toxic solvents include water and ethanol.

In one extraction method which can be used herein, the plant portion to be extracted is placed into an extractor, 70% ethanol is added, and the resultant mixture is heated under reflux. Ethanol is recovered and condensed under low temperature and decompression until the specific density reaches 1.38 (thermal assay). The extract is then collected by vacuum drying.

The herbal composition of this invention can be prepared, for example, by individually washing, drying and grinding the ginger and rosemary herbs into fine powder, and then subjecting the ground ginger to supercritical extraction and the rosemary to either supercritical or regular extraction. The resulting extracts are then mixed together in amounts that are physiologically acceptable to the patient. No special mixing means is required. The mixture of extracts can be encapsulated, tableted or formulated with a physiologically acceptable vehicle into unit dosages.

The herbal composition of this invention contains therapeutically effective amounts of the ginger and rosemary extracts discussed above. As used herein, the term “therapeutically effective amount” with respect to the ginger and rosemary extracts means those amounts of the extracts that will promote stomach, liver and intestinal health, reduce inflammation, support blood platelet health and cardiovascular function, and provide antioxidant benefits.

The herbal composition of this invention can be administered orally or parenterally (e.g., by intravenous drip or by intraperitoneal, subcutaneous or intramuscular injection). Most preferably, the composition of this invention is administered orally.

The orally administered embodiments of the herbal composition of this invention can be in any conventional form such as, e.g., capsules (hard or soft), tablets, elixirs, powders, granules, suspensions in water or non-aqueous media, sachets, as additives to food or beverages, or even can be made into a tea. Most preferably, the orally administered embodiment of the composition is in the form of a soft gel capsule which is swallowed with water.

For preparing solid orally administered compositions such as capsules or tablets, the principal active ingredients are mixed with a pharmaceutical carrier (e.g., conventional tableting ingredients such as corn starch, lactose, sucrose, sorbitol, talc, stearic acid, magnesium stearate, dicalcium phosphate or gums) and other pharmaceutical diluents (e.g., water) to form a solid preformulation composition containing a substantially homogenous mixture of the composition of this invention, or a non-toxic pharmaceutically acceptable salt thereof. When referring to the preformulation compositions as substantially homogenous, it is meant that the active ingredients are dispersed evenly throughout the composition so that the composition may be readily subdivided into equally effective unit dosage forms such as capsules, pills and tablets. This solid preformulation composition can then be subdivided into unit dosage forms containing, for example, from 0.15 to 1.0 gram, of the active-ingredient composition of this invention.

Liquid preparations for oral administration may take the form of, for example, solutions, syrups or suspensions, or they may be presented as a dry product for reconstitution with water or other suitable vehicles before use. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

The active compounds may be formulated for parenteral administration by injection, which includes using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as stabilizing, suspending or dispersing agents. Alternatively, the active ingredients may be in powder form for reconstitution with a suitable vehicle, e.g., sterile pyrogert-free water, before use.

The herbal composition of this invention can be combined with the physiologically acceptable vehicle into unit dosages. A unit dosage can comprise a therapeutically effective amount of each herbal extract for a single daily administration (e.g., orally), or it can be formulated into smaller quantities of each ingredient to provide for multiple doses in a day. A unit dosage will depend upon many factors including age, size, and condition of the patient being treated and the number of times the unit will be taken in a single day. In any event, the entire daily dosage will be that which is physiologically acceptable to an individual and can be administered daily over a prolonged period of time. In the present invention, normally between about 100 and 2000 mg of the active herb composition is orally administered per day, with part of the total dose preferably taken at two or more different times during the day.

The exact proportion of the two extracts used in the composition of this invention will depend on the concentration of active ingredients found naturally in each extract. Using the guidance provided herein and a basic knowledge of drug preparation and pharmacology, one skilled in the art could easily adjust the proportions of the separate components of the composition so as to obtain a composition which has the therapeutic effects discussed herein.

The present invention is also directed to methods of promoting stomach, liver and intestinal health; reducing inflammation; supporting cardiovascular function; and providing antioxidant activity, using the herbal active-ingredient composition of this invention. The methods involve orally or parenterally administering an effective amount of the active-ingredient composition to an individual in need of the aforementioned therapeutic benefits. As used herein, the term “effective amount” with respect to the active-ingredient composition means that amount sufficient to provide the above-recited therapeutic benefits. The effective amount will depend upon the severity of the symptoms and on the responsiveness of the patient to the herbal composition. Persons of ordinary skill in the art can easily determine optimum dosages, dosing methodologies, and repetition rates.

Oral administration of the active-ingredient composition involves ingesting the composition. As stated previously herein, the most preferred form of orally administered composition of the present invention is the soft gel capsule, which is preferably swallowed with water.

Suitable modes of parenteral administration include, e.g., intravenous dip, intraperitoneal, subcutaneous, or intramuscular injection; and the like.

Presented in the table below is a particularly preferred embodiment of the orally administered soft gel capsule form of the composition of this invention. The formulation below represents the composition of a single soft gel capsule. In other words, one capsule constitutes a single serving or unit dose of the composition.

TABLE
Herbal Active-Ingredient Composition: Formulation
(One Soft Gel Capsule)

| | Amount |
|---|---|
| Ingredient | (milligrams) |
| Ginger, rhizome, certified organic, supercritical extract | 100 |
| (minimum 20% pungent compounds-20 mg, | |
| 5% zingiberene-5 mg) | |
| Rosemary, leaf & essential oil, supercritical extract | 5 |
| (23% total phenolic antioxidants [TPA]-1.15 mg) | |

The herbal composition set forth in the table above preferably further contains olive oil (certified organic) and yellow beeswax.

The soft gel capsule is preferably composed of gelatin, vegetable glycerine, purified water and carob.

For oral administration of the above-recited formulation, one to three of the soft gel capsules should be taken daily, with 8 ounces of water.

What is claimed is:

1. A ginger-based herbal composition for promoting health, comprising therapeutically effective amounts of a supercritical extract of ginger and a regular or supercritical extract of rosemary.

2. A composition according to claim 1, wherein the ginger is certified organic ginger.

3. A composition according to claim 2, wherein the supercritical ginger extract comprises at least 20% by weight of pungent gingerols and about 5% by weight of zingiberene.

4. A composition according to claim 3, wherein the rosemary extract is a supercritical rosemary extract comprising carnosic acid.

5. A composition according to claim 4, wherein the rosemary supercritical extract comprises about 23% by weight of phenolic antioxidants.

6. A composition according to claim 1, wherein the supercritical extract of the ginger is a supercritical rhizome extract of the ginger, and the regular or supercritical extract of the rosemary is a leaf and/or essential oil regular or supercritical extract of the rosemary.

7. A composition according to claim 1, wherein the composition is an orally administered or parenterally administered composition.

8. A composition according to claim 7, wherein the composition is an orally administered composition selected from the group consisting of capsules, tablets, elixirs, powders, granules, suspensions, sachets, food additives, beverage additives, and tea.

9. A composition according to claim 8, wherein the orally administered composition is in the form of one or more soft gel capsules.

10. A composition according to claim 9, wherein each of the soft gel capsules comprises about 100 milligrams of a supercritical extract of certified organic ginger rhizome and about 5 milligrams of a supercritical extract of rosemary leaf and essential oil; said about 100 milligrams of the supercritical ginger extract comprising at least 20% by weight of pungent gingerol compounds and about 5% zingiberene; the about 5 milligrams of the supercritical rosemary extract comprising about 23% by weight of phenolic antioxidants.

11. A method for promoting health in a person, comprising administering to said person a ginger-based herbal composition comprising therapeutically effective amounts of a supercritical extract of ginger and a regular or supercritical extract of rosemary.

12. A method according to claim 11, wherein the ginger is certified organic ginger.

13. A method according to claim 12, wherein the supercritical ginger extract comprises at least 20% by weight of pungent gingerols and about 5% by weight of zingiberene.

14. A method according to claim 13, wherein the rosemary extract is a supercritical rosemary extract comprising carnosic acid.

15. A method according to claim 14, wherein the rosemary supercritical extract comprises about 23% by weight of phenolic antioxidants.

16. A method according to claim 11, wherein the supercritical extract of the ginger is a supercritical rhizome extract of the ginger, and the regular or supercritical extract of the rosemary is a leaf and/or essential oil regular or supercritical extract of the rosemary.

17. A method according to claim 11, comprising orally or parenterally administering the composition to said person.

18. A method according to claim 11, wherein the composition is an orally administered composition selected from the group consisting of capsules, tablets, elixirs, powders, granules, suspensions, sachets, food additives, beverage additives, and tea.

19. A method according to claim 18, wherein the orally administered composition is in the form of one or more soft gel capsules.

20. A method according to claim 19, wherein each of the soft gel capsules comprises about 100 milligrams of a supercritical extract of certified organic ginger rhizome and about 5 milligrams of a supercritical extract of rosemary leaf and essential oil; said about 100 milligrams of the supercritical ginger extract comprising at least 20% by weight of pungent gingerol compounds and about 5% zingiberene; the about 5 milligrams of the supercritical rosemary extract comprising about 23% by weight of phenolic antioxidants.

21. A method according to claim 20, comprising orally administering one to three of said soft gel capsules to said person on a daily basis.
